(19) United States
(12) Patent Application Publication
(10) Pub. No.: US 2022/0062172 A1
(43) Pub. Date: Mar. 3, 2022

(54) TOPICAL FORMULATION AND METHODS FOR PAIN RELIEF

(71) Applicant: Jason Pool, Horseshoe Bay, TX (US)

(72) Inventor: Jason Pool, Horseshoe Bay, TX (US)

(21) Appl. No.: 17/338,248

(22) Filed: Jun. 3, 2021

Related U.S. Application Data
(60) Provisional application No. 63/069,839, filed on Aug. 25, 2020.

(51) Int. Cl. A61K 9/06; A61K 31/196; A61K 31/167; A61K 9/00; A61K 31/135; A61K 31/05; A61K 47/10

(52) U.S. Cl. CPC A61K 9/06; A61K 31/196; A61K 31/167; A61K 47/10; A61K 31/135; A61K 31/05; A61K 9/0014

(57) ABSTRACT

Disclosed herein is a topical formulation for relief of pain and inflammation. The formulation comprises diclofenac sodium and lidocaine HCL in a cream or ointment base for topical application for relief of pain and inflammation. Disclosed herein is a method of making a formulation for relief of pain and inflammation, the formulation comprising diclofenac sodium and lidocaine HCL in a cream or ointment base suitable for topical application. Disclosed herein is a method of using a formulation for relief of pain and inflammation, the formulation comprising diclofenac sodium and lidocaine HCL in a cream or ointment base for topical application to relieve pain and inflammation.

### CROSS REFERENCE TO RELATED APPLICATIONS

This application claims the benefit of U.S. Provisional Application No. 63/069,839 filed Aug. 25, 2020, which is incorporated by reference into this utility patent application.

### STATEMENT REGARDING FEDERALLY SPONSORED RESEARCH OR DEVELOPMENT

Not applicable.

### TECHNICAL FIELD OF INVENTION

This invention relates to the field of topical treatment of musculoskeletal pain.

### BACKGROUND OF THE INVENTION

Pain is a signal in the nervous system caused by damage or intense stimuli in the body and inflammation is part of the body's process of fighting against the damage. Musculoskeletal pain, affecting the bones, muscles, ligaments, tendons, nerves and surrounding tissues, is a common type of pain, and is typically accompanied by inflammation in the affected area. This type of pain can be acute, having a rapid onset, or chronic, of long-lasting duration. Musculoskeletal pain can have many causes, including injury, overuse, infection, arthritis, and auto-immune disorders, to name but a few. Regardless of the cause, if pain and inflammation occur, it is desirable to have a way to provide relief.

Oral medications for pain and inflammation frequently produce unwanted side-effects, such as gastro-intestinal problems or drowsiness. Often, the severity of the side-effects mean that a choice may have to be made between the pain and the remedy.

Many unwanted side-effects produced by oral pain medications can be bypassed using a different type of administration. Not all pain medications can be absorbed in therapeutically useful dosages when applied by other types of administration. What is needed is a pain relief formulation that can be successfully delivered by another route of administration and effectively relieve pain and inflammation from numerous causes.

### SUMMARY OF THE INVENTION

Disclosed herein is a pain relief formulation for topical application to relieve pain and inflammation. Disclosed herein is a method of making a pain relief formulation for topical application to relieve pain and inflammation. Disclosed herein is a method of using a pain relief formulation for treatment of pain and inflammation.

Disclosed herein is a method of making a formulation, the method comprising the steps of: providing therapeutic agents for topical application, the therapeutic agents comprising diclofenac sodium and lidocaine HCL; providing a penetration-enhancing humectant; combining the diclofenac sodium, lidocaine HCL, and the penetration-enhancing humectant to form a substantially homogenous paste of therapeutic agents; providing one or more pharmaceutically suitable carrier suitable for topical application of the therapeutic agents, the pharmaceutically suitable carrier having at least one emulsifying agent; combining the substantially homogenous paste of therapeutic agents with the one or more pharmaceutically suitable carrier to produce a combined substantially homogeneous paste of therapeutic agents; and processing the combined substantially homogenous paste of therapeutic agents and the one or more pharmaceutically suitable carrier in an ointment mill to produce a substantially homogenous topical formulation having therapeutic concentrations of diclofenac sodium and lidocaine HCL.

The substantially homogenous topical formulation has diclofenac sodium 1% to 25% w/w and lidocaine 1-25% w/w in one or more pharmaceutically suitable carrier. The substantially homogenous topical formulation may have diclofenac sodium 5% w/w and lidocaine 5% w/w in one or more pharmaceutically suitable carrier. The substantially homogenous topical formulation may have diclofenac sodium 3% w/w and lidocaine 5% w/w in a pharmaceutically suitable carrier.

The therapeutic agents may further comprise 5%-15% w/w ketoprofen. The therapeutic agents may further comprise 2%-10% w/w ketamine HCL. The therapeutic agents may further comprise 2%-10% w/w menthol. The substantially homogenous topical formulation may have one or more of ketoprofen 5%-10% w/w, ketamine HCL 2%-5% w/w, and menthol 2%-10% w/w.

The pharmaceutically suitable carrier comprises one or more of a humectant, penetrant, protectant, emollient, surfactant, and moisturizing ingredient suitable for topical delivery of pharmaceutical therapeutics. An exemplary penetration-enhancing humectant is propylene glycol.

The formulation is a topical treatment for musculoskeletal pain. The substantially homogenous topical formulation may be applied to an affected area one, two, three or four times a day for treatment of musculoskeletal pain.

Disclosed herein is a formulation comprising: diclofenac sodium, lidocaine HCL and a penetration-enhancing humectant in a pharmaceutically suitable carrier for topical application. The disclosed formulation has 2% to 10% w/w diclofenac sodium. The disclosed formulation has 2% to 10% w/w lidocaine HCL. The disclosed formulation may further comprise one or more of ketoprofen 5%-15% w/w, ketamine HCL 2%-10% w/w, and menthol 2%-10% w/w.

The pharmaceutically suitable carrier for the formulation has at least one emulsifying agent. The pharmaceutically suitable carrier has one or more of a humectant, penetrant, protectant, emollient, preservative, and moisturizing ingredient suitable for topical delivery of pharmaceutical therapeutics.

The formulation is a topical treatment for musculoskeletal pain. The formulation may be applied topically to an affected area one, two, three or four times a day for treatment for musculoskeletal pain.

Disclosed herein is a topical formulation comprising: 2% to 10% w/w lidocaine HCL; 2% to 10% w/w diclofenac sodium; a penetration-enhancing humectant; and a pharmaceutically suitable carrier having at least one emulsifying agent and one or more of a humectant, penetrant, protectant, emollient, surfactant, and moisturizing ingredient suitable for topical delivery of pharmaceutical therapeutics.

In the disclosed topical formulation, the penetration-enhancing humectant is propylene glycol. In the disclosed topical formulation, the concentration of diclofenac sodium may be from 3% to 5% w/w. In the disclosed topical formulation, the concentration of lidocaine HCL may be from 3%-5% w/w. In the disclosed topical formulation, the concentration of diclofenac sodium may be from 3% to 5% w/w and the lidocaine HCL may be from 3%-5% w/w.

The topical formulation is a topical treatment for musculoskeletal pain. The topical formulation may be applied topically to an affected area one, two, three or four times a day for treatment for musculoskeletal pain.

### DETAILED DESCRIPTION OF THE INVENTION

Disclosed herein is a topical formulation and methods for relief of pain and inflammation. Disclosed herein is a method of making a formulation for topical application to relieve pain and inflammation. Disclosed herein is a method of using a topical formulation for relief of pain and inflammation. In a preferred embodiment for topical application to the skin, the disclosed formulation comprises a synergistic mixture of diclofenac sodium and lidocaine HCL in a pharmaceutically suitable carrier for relief of pain and inflammation.

The disclosed pain relief formulation comprises a synergistic combination of diclofenac sodium and lidocaine HCL in a pharmaceutically suitable carrier for topical application to an area in need of treatment. The disclosed analgesic and anti-inflammatory formulation is useful for all types of musculoskeletal pain regardless of the source, including pain or inflammation associated with bones, tissues, muscles, or joints. The pain relief formulation is applied topically to an affected area three to four times daily for relief of pain. The formulation may also comprise one or more of ketoprofen, ketamine and menthol.

Diclofenac sodium is a nonsteroidal anti-inflammatory drug (NSAID) that works by reducing the production of prostaglandins in the body that cause pain and inflammation. The disclosed formulation has 1% to 25% w/w diclofenac sodium. The disclosed formulation may have 2% to 10% w/w diclofenac sodium. The disclosed formulation may have 3% to 5% w/w diclofenac sodium. A 3% concentration of diclofenac sodium is recommended for mild to moderate pain. A 5% concentration of diclofenac sodium is recommended for severe or chronic pain.

Lidocaine is a local anesthetic that can be used to relieve pain and itching. The medication works by causing temporary numbness or loss of feeling in the area where applied. The disclosed formulation has 1% to 20% w/w lidocaine HCL. The disclosed formulation may have 2% to 10% Lidocaine HCL. The disclosed formulation may have 3% to 5% Lidocaine HCL. A 5% concentration of lidocaine HCL is recommended for moderate to severe pain.

The diclofenac sodium and lidocaine HCL are combined with one or more pharmaceutically suitable carrier compatible with hydrophobic and hydrophilic therapeutic actives to produce a smooth cream or ointment formulation for topical administration. Pharmaceutically suitable carriers will utilize one or more emulsifying agent to stably disperse lipid and aqueous components and is used in combination with pharmaceutically acceptable humectant, penetrant, protectant, emollient, surfactant and/or moisturizing ingredients suitable for topical delivery of pharmaceutical therapeutic agents. Pharmaceutically suitable carrier components include one or more of alcohols, diols, fatty acids, fatty acid esters, fatty alcohols, oils, glycols, polyols, behenyl alcohol, butylene glycol, caprylic acids, cetyl alcohol, cetearyl alcohol, glycerides, glycerin, hexylene glycol, methylpropane diol, mineral oils, petrolatum, propylene glycol, sodium laureth sulfate, steric acid, stearyl alcohol, urea, vegetable oils. Additional excipient ingredients may be included in the pharmaceutically suitable carrier, such as stabilizing, solubilizing, and preservative ingredients.

Disclosed is a formulation having a synergistic combination of diclofenac sodium 1% to 25% w/w and lidocaine HCL 1% to 20% w/w in a pharmaceutically suitable carrier for topical application. In one embodiment for mild to moderate pain, the formulation has 3% diclofenac sodium and 5% lidocaine HCL. In another embodiment for moderate to severe pain, the formulation has 5% diclofenac sodium and 5% lidocaine HCL.

The topical formulation may include one or more of ketoprofen, ketamine HCL and menthol as additional therapeutic agents.

Ketoprofen is a nonsteroidal anti-inflammatory drug (NSAID) that possesses analgesic, antipyretic and anti-inflammatory properties. Ketoprofen has been used topically to treat musculoskeletal pain and inflammation in muscles and joints, including osteoarthritis and injuries. The disclosed formulation may have 5% to 30% w/w ketoprofen. The formulation may have 5% to 15% w/w ketoprofen. The formulation may have 7.5% to 10% w/w ketoprofen.

Ketamine HCL is an anesthetic agent that can be used topically to treat musculoskeletal pain. The disclosed formulation may have 1% to 20% w/w ketamine HCL. The formulation may have 2% to 10% w/w ketamine HCL. The formulation may have 2% to 5% w/w ketamine HCL.

Menthol is an organic compound made synthetically or obtained from the oils of peppermint or other mint plant varieties. Menthol has mild analgesic properties and has a cooling effect on tissues which acts as a counter-irritant. The disclosed formulation may have 1% to 20% w/w menthol. The formulation may have 2% to 10% w/w menthol.

### Exemplary Preparation of Pain Relief Formulation

To prepare the disclosed pain relief formulation, effective amounts of diclofenac sodium powder and lidocaine HCL powder are combined and triturated with a penetration-enhancing humectant to make a substantially uniform paste. Trituration with a mortar and pestle can be used to grind the active ingredients to a substantially uniform paste. A powder spatula and tile can be used to grind the active ingredients to a substantially uniform paste. A blade grinder can be used to grind the active ingredients to a substantially uniform paste. A manual or mechanical tumbler can be used to produce a substantially uniform paste mixture of the active ingredients. One example of a penetration-enhancing humectant is propylene glycol, however other equivalent penetration-enhancing humectants suitable to form a paste with the therapeutic agents, including but not limited to sulphoxides, pyrrolidones, alcohols and alkanols, glycols, surfactants, and terpenes, may be used instead of or in addition to the propylene glycol. A substantially uniform paste is also referenced as a substantially homogeneous paste. By substantially uniform or substantially homogeneous, the composition or paste would be a substantially uniform consistency with 85%-100% fully mixed to meet that substantial uniformity.

The paste of therapeutic agents is combined with a pharmaceutically suitable carrier compatible with hydrophobic and hydrophilic therapeutic actives, stirring until the mixture is homogenous. An emulsifying concentrate utilizes one or more emulsifying agent to stably disperse lipid and aqueous components and is used in combination with pharmaceutically acceptable humectant, penetrant, protectant, emollient, surfactant, and/or moisturizing ingredients that are suitable for topical delivery of pharmaceutical therapeutics. Additional excipient ingredients may be included in the pharmaceutically suitable carrier, such as stabilizing, solubilizing, and preservative ingredients. Pharmaceutically suitable carriers may be an individual carrier component, combinations of individual carrier components, or one or more commercially available carrier combinations. An example of a commercially available carrier combination of an emulsifying concentrate is Humco™ emulsion concentrate, however other suitable types of emulsion concentrate or combinations of emulsion concentrates may also be used. An example of a commercially available carrier combination for an emollient base is LetcoTM emollient cream base, however other suitable types of emollient cream base or combinations of emollient cream or ointment bases may be used.

Once diclofenac sodium, lidocaine HCL, propylene glycol, emulsifying concentrate and emollient base are uniformly combined to a homogeneously cream-type mixture, the resulting cream-type mixture is processed by a mechanical incorporation procedure to produce a smooth formulation with therapeutic agents uniformly distributed therein. The resulting cream is processed with an ointment mill or a blade mixer to produce a substantially uniformly smooth final composition. The diclofenac sodium, lidocaine HCL, propylene glycol, emulsifying concentrate and emollient base are processed through an ointment mill to reduce the particle sizes of the active ingredients and produce a substantially uniformly smooth final composition. A roller-type ointment mill uses high shear force to reduce and disperse the particles in the cream base to produce a substantially homogenous composition.

An exemplary milling method processes the cream through the ointment mill at least two times. The roller mill gaps are set to balance throughput of the material through the mill with the desired dispersion of the particles in the composition. An exemplary setting for the roller mill is a gap setting of 2 in the front and 2 in the back. When the pain relief formulation is fully milled, the finished product is dispensed in 1, 2, or 3 ounce metal tubes, pursuant to prescription requirements.

### Exemplary Embodiments of the Pain Relief Cream

The pain relief formulation has 1%-25% concentration of diclofenac sodium and a 1%-25% concentration of lidocaine HCL in a pharmaceutically suitable carrier. The pharmaceutically suitable carrier includes one or more penetration-enhancing humectant, a concentrated emulsifying agent and a emollient base, and any combinations thereof. The exemplary formulations are examples only with higher and lower concentrations of the diclofenac sodium and lidocaine HCL envisioned, depending on the treatment required.

One embodiment of the pain relief formulation has a 3% concentration of diclofenac sodium and a 5% concentration of lidocaine HCL in an emollient base for topical administration. This embodiment is useful for relief of mild to moderate pain.

Another embodiment of the pain relief formulation has a 5% concentration of diclofenac sodium and a 5% concentration of lidocaine HCL in an emollient base for topical administration. This embodiment is useful for relief of severe or chronic pain.

An exemplary embodiment of the pain relief formulation for relief of severe or chronic pain comprises the active ingredients in the following concentrations: 5% concentration of diclofenac sodium and a 5% concentration of lidocaine HCL. An exemplary formulation for relief of severe or chronic pain is shown in Table 1 with the active ingredients combined with penetration-enhancing humectant, an emulsifying concentrate and an emollient base suitable for topical application of pharmaceutical active ingredients.

TABLE 1

| Active Ingredients (Final | |
|---|---|
| Concentration % w/w) | Amount |
| Diclofenac sodium USP (5%) | 45 grams |
| Lidocaine HCL USP (5%) | 45 grams |
| | 90 grams (total actives) |
| Inactive Compounding Ingredients | |
| Propylene glycol USP | 10 mL |
| Emulsifying Concentrate | 45 grams |
| Emollient Base | 755 grams (add to other |
| | ingredients to q.s. to 900 |
| | grams) |

To prepare 900 grams of the exemplary pain relief formulation, 45 grams of diclofenac sodium powder and 45 grams of lidocaine HCL powder are combined and triturated with 10 mL of a penetration-enhancing humectant to make a substantially uniform paste. Trituration with a mortar and pestle can be used to grind the active ingredients to a substantially uniform paste. A powder spatula and tile can be used to grind the active ingredients to a substantially uniform paste. A blade grinder can be used to grind the active ingredients to a substantially uniform paste. A manual or mechanical tumbler can be used to produce a substantially uniform paste mixture of the active ingredients. An example of a penetration-enhancing humectant is propylene glycol, however penetration-enhancing humectants suitable to form a paste with the therapeutic agents, including sulphoxides, pyrrolidones, alcohols and alkanols, glycols, surfactants, and terpenes, and may be used instead of or in addition to the propylene glycol.

The resulting substantially uniform paste of active ingredients is combined with one or more pharmaceutically suitable carrier compatible with hydrophobic and hydrophilic therapeutic agents and stirred until the mixture is substantially homogenous. In the exemplary process, the paste is mixed with 45 grams of a concentrated emulsifying agent and 755 grams of an emollient base and mixed until a substantially homogenous cream-type mixture results.

An emulsifying concentrate utilizes one or more emulsifying agent to stably disperse lipid and aqueous components and is used in combination with pharmaceutically acceptable humectant, penetrant, protectant, emollient, surfactant, and/or moisturizing ingredients that are suitable for topical delivery of pharmaceutical therapeutics. Additional excipient ingredients may be included in the pharmaceutically suitable carrier, such as stabilizing, solubilizing, and preservative ingredients. Pharmaceutically suitable carriers may be an individual carrier component, combinations of individual carrier components, or one or more commercially available carrier combinations. An example of a commercially available carrier combination of an emulsifying concentrate is Humco™ emulsion concentrate, however other types of emulsion concentrate or combinations of emulsion concentrates may also be used instead of or in combination with this emulsion concentrate. An example of a commercially available carrier combination for an emollient base is Letco™ emollient cream base, however other types of emollient cream base or combinations of emollient cream or ointment bases may also be used instead of or in combination with this emollient base.

Once the diclofenac sodium, lidocaine HCL, propylene glycol, emulsifying concentrate and emollient base are homogeneously combined, the resulting cream is processed by a mechanical incorporation procedure to produce a smooth formulation with therapeutic agents substantially uniformly distributed therein. The resulting cream is processed with an ointment mill or a blade mixer to produce a substantially uniformly smooth final composition. The resulting cream is processed through an ointment mill to reduce the particle sizes of the active ingredients and produce a substantially uniformly smooth final composition. A roller-type ointment mill uses high shear force to reduce and disperse the particles in the cream base to produce a homogenous composition. In an exemplary milling process, the cream is processed through the ointment mill at least two times. The roller mill gaps are set to balance throughput of the material through the mill with the desired dispersion of the particles in the composition. An exemplary setting for the roller mill is a gap setting of 2 in the front and 2 in the back. When the pain relief formulation is fully milled, the finished product is dispensed in 1, 2, or 3 ounce metal tubes, pursuant to prescription requirements.

The exemplary formulation is an example of a small-scale processing of the formulation; however, large scale processing of the formulation is also envisioned. Large scale processing will proceed in a manner similar to the small-scale process with ingredients proportionally scaled-up for volume processing to produce a substantially smooth final composition with therapeutic actives substantially uniformly distributed in the formulation. Large-scale processing would utilize mechanical processing equipment known in the art for production of topical creams or ointments.

Embodiments of the pain relief formulation within the scope of this disclosure may have concentrations of the diclofenac sodium and lidocaine HCL, that differ from the exemplary embodiment in Table 1. In embodiments having the therapeutic in concentrations that differ from the exemplary embodiment, the quantity of emollient base is adjusted proportionally in order to maintain the desired concentration of the therapeutic agents. Preparations of the pain relief formulation with more or less than the 5% concentration of diclofenac sodium and lidocaine HCL in the above example will be made in the same manner. If concentrations of the therapeutic agents are increased, the amount of emollient base will be decreased proportionally to achieve the desired concentration of the therapeutic agents. If concentrations of the active ingredients decrease, the amount of emollient base will increase proportionally to achieve the desired concentration of the therapeutic agents.

The pain relief formulation may have one or more of ketoprofen, ketamine HCL or menthol, in addition to the diclofenac sodium and lidocaine HCL. In an embodiment where the formulation incorporates one or more of the optional therapeutic agents, preparation of the pain relief formulation will be same as for a formulation having only diclofenac sodium and lidocaine HCL as therapeutic agents. In an embodiment of the formulation including ketoprofen, ketamine HCL or menthol, or combinations thereof, the amount of emulsifying concentrate and/or the emollient base will be adjusted proportionally in order to maintain the desired concentration of the diclofenac sodium and lidocaine HCL therapeutic agents.

### Exemplary Use of Pain Relief Formulation

The pain relief formulation is applied in an amount sufficient to cover the affected area of clean dry skin. The pain relief formulation is rubbed into the affected area until no cream remains visible on the surface. The formulation can be applied one, two, three or four times per day as needed for relief of symptoms.

Significant benefits of the cream have been seen when used for musculoskeletal pain of many origins, including, but not limited to, osteoarthritis, muscle strains, muscle spasms, muscle sprains, and tendonitis. The combination of diclofenac sodium and lidocaine HCL in the pain relief formulation has been found to work synergistically to alleviate pain more effectively than the known individual therapeutics. The following example cases are from a few of the individuals who have been successfully treated with the claimed pain relief formulation.

### Example 1

Patient DP is a secretary and was experiencing severe joint pain in several fingers on both hands. The joint pain interfered with the ability to perform job-related tasks such as typing. Oral pain medications had been used daily to relieve the pain, but caused problematic side-effects, including constant stomach pain and other gastro-intestinal symptoms. DP began using the disclosed pain relief formulation and had a substantial reduction in pain levels. DP no longer requires daily oral pain medication to control the severe joint pain.

### Example 2

Patient DC is a golfer who was experiencing chronic knee pain. The knee pain caused DC to give up golfing. DC began using the disclosed pain relief formulation and the pain quickly decreased such that DC was able to return to the driving range within three days of beginning therapy. DC has since been able to return to playing full rounds of golf.

### Example 3

Patient JL is the owner of a paving company and had been experiencing severe muscle pain across the shoulders and upper back for about one year. Acetaminophen and ibuprofen were used regularly by the patient to alleviate pain enough to sleep. JL began using the disclosed pain relief formulation and experienced significant relief of the pain. JL reported that pain relief begins within 10 minutes of application of the formulation and JL no longer has to depend on acetaminophen and ibuprofen for relief of the pain.

1. A method of making a formulation, the method comprising the steps of:
providing therapeutic agents for topical application, said therapeutic agents comprising diclofenac sodium and lidocaine HCL;
providing a penetration-enhancing humectant;
combining said diclofenac sodium, lidocaine HCL, and said penetration-enhancing humectant to form a substantially homogenous paste of therapeutic agents;
providing one or more pharmaceutically suitable carrier suitable for topical application of said therapeutic agents, said pharmaceutically suitable carrier having at least one emulsifying agent;
combining the substantially homogenous paste of therapeutic agents with said one or more pharmaceutically suitable carrier to produce a combined substantially homogeneous paste of therapeutic agents; and
processing the combined substantially homogenous paste of therapeutic agents and said one or more pharmaceutically suitable carrier in an ointment mill to produce a substantially homogenous topical formulation having therapeutic concentrations of diclofenac sodium and lidocaine HCL.

2. The method of claim 1, wherein the substantially homogenous topical formulation has diclofenac sodium 1% to 25% w/w and lidocaine 1-25% w/w in said one or more pharmaceutically suitable carrier.

3. The method of claim 2, wherein the substantially homogenous topical formulation has diclofenac sodium 5% w/w and lidocaine 5% w/w in said one or more pharmaceutically suitable carrier.

4. The method of claim 2, wherein the substantially homogenous topical formulation has diclofenac sodium 3% w/w and lidocaine 5% w/w in a pharmaceutically suitable carrier.

5. The method of claim 1, wherein the therapeutic agents further comprise 5%-15% w/w ketoprofen.

6. The method of claim 1, wherein the therapeutic agents further comprise 2%-10% w/w ketamine HCL.

7. The method of claim 1, wherein the therapeutic agents further comprise 2%-10% w/w menthol.

8. The method of claim 2, wherein the substantially homogenous topical formulation has one or more of ketoprofen 5%-10% w/w, ketamine HCL 2%-5% w/w, and menthol 2%-10% w/w.

9. The method of claim 1, wherein said pharmaceutically suitable carrier comprises one or more of a humectant, penetrant, protectant, emollient, surfactant, and moisturizing ingredient suitable for topical delivery of pharmaceutical therapeutics.

10. The method of claim 1, wherein the penetration-enhancing humectant is propylene glycol.

11. The method of claim 1, wherein the formulation is a topical treatment for musculoskeletal pain.

12. The method of claim 10, wherein the substantially homogenous topical formulation is applied to an affected area one, two, three or four times a day for treatment of musculoskeletal pain

13. A formulation comprising: diclofenac sodium, lidocaine HCL and a penetration-enhancing humectant in a pharmaceutically suitable carrier for topical application.

14. The formulation of claim 13, having 2% to 10% w/w diclofenac sodium.

15. The formulation of claim 13, having 2% to 10% w/w lidocaine HCL.

16. The formulation of claim 13, further comprising one or more of ketoprofen 5%-15% w/w, ketamine HCL 2%-10% w/w, and menthol 2%-10% w/w.

17. The formulation of claim 13, wherein said pharmaceutically suitable carrier has at least one emulsifying agent.

18. The formulation of claim 13, wherein said pharmaceutically suitable carrier has one or more of a humectant, penetrant, protectant, emollient, preservative, and moisturizing ingredient suitable for topical delivery of pharmaceutical therapeutics.

19. The formulation of claim 13, wherein the formulation is a topical treatment for musculoskeletal pain.

20. The formulation of claim 19, wherein the formulation is applied topically to an affected area one, two, three or four times a day for treatment for musculoskeletal pain.

21. A topical formulation comprising:
2% to 10% w/w lidocaine HCL;
2% to 10% w/w diclofenac sodium;
a penetration-enhancing humectant; and
a pharmaceutically suitable carrier having at least one emulsifying agent and one or more of a humectant, penetrant, protectant, emollient, surfactant, and moisturizing ingredient suitable for topical delivery of pharmaceutical therapeutics.

22. The topical formulation of claim 21, wherein said penetration-enhancing humectant is propylene glycol.

23. The topical formulation of claim 21, wherein the concentration of diclofenac sodium is from 3% to 5% w/w.

24. The topical formulation of claim 21, wherein the concentration of lidocaine HCL is from 3%-5% w/w.

25. The topical formulation of claim 21, wherein the concentration of diclofenac sodium is from 3% to 5% w/w and the lidocaine HCL is from 3%-5% w/w.

26. The topical formulation of claim 21, further comprising 5%-15% w/w ketoprofen.

27. The topical formulation of claim 21, further comprising 2%-10% w/w ketamine HCL.

28. The topical formulation of claim 21, further comprising 2%-10% w/w menthol.

29. The topical formulation of claim 21, wherein the topical formulation is a topical treatment for musculoskeletal pain.

30. The topical formulation of claim 29, wherein the formulation is applied topically to an affected area one, two, three or four times a day for treatment for musculoskeletal pain.
